(19) United States
(12) Patent Application Publication
(10) Pub. No.: US 2011/0097754 A1
(43) Pub. Date: Apr. 28, 2011

(54) METHOD FOR MEASURING ACTIVATED FACTOR VII LEVEL IN A SAMPLE

(75) Inventors: Lysiane Hilbert, Villeneuve-d'Asoq (FR); Claudin Mazurier, Villeneuve-d'Ascq (FR); Dominique Grenier, Hellemmes (FR)

(73) Assignee: LFB BIOTECHNOLOGIES, Les Ulis (FR)

(21) Appl. No.: 13/002,020

(22) PCT Filed: Jun. 29, 2009
(86) PCT No.: PCT/IB2009/006099

§ 371 Date: Dec. 29, 2010

(30) Foreign Application Priority Data
Jul. 2, 2008 (FR) 0803743

(51) Int. Cl. C12Q 1/56

(52) U.S. Cl. 435/13

(57) ABSTRACT

The present disclosure relates to a method for measuring the activated factor VII level in a sample to be tested, including the steps of: a) mixing the test sample with a plasma free of factor VII (FVII) and free of at least another factor selected from among factor VIII (FVIII), factor IX (FIX), and factor XI (FXI), the test sample+plasma having a final FVII+FVIIa concentration of 10 pM to 80 pM; b) adding initiating components from the thrombin generation reaction; c) obtaining a thrombogram when carrying out a thrombin generation test (TGT) on the mixture from step b); d) comparing at least one of the thrombogram parameters from step c) with a homologous parameter obtained from standard thrombograms established on the basis of standard samples, the activated factor VII level of which is known and varies with each standard sample; e) deducing, from step d), an activated factor VII level measurement in the test sample.

### CROSS-REFERENCE TO RELATED APPLICATIONS

This application is a National Phase Entry of International Application No. PCT/IB2009/006099, filed on Jun. 29, 2009, which claims priority to French Application 0803743, filed on Jul. 2, 2008, both of which are incorporated by reference herein.

### TECHNICAL FIELD

The present invention relates to a method for measuring the level of activated Factor VII (FVIIa) in a sample using a plasma that is deficient in FVII and in at least one other factor chosen from Factor VIII (FVIII), Factor IX (FIX) and Factor XI (FXI).

### BACKGROUND

Blood coagulation is a mechanism which allows organisms to control bleeding when vascular lesions occur and thus to avoid hemorrhaging. Blood coagulation takes place following a cascade of steps involving different proenzymes and procofactors present in the blood which are converted, via proteolytic enzymes, to their activated form. In this succession of steps (or cascade) of coagulation, two pathways are distinguished, called the extrinsic coagulation pathway and the intrinsic coagulation pathway. Both lead to the formation of the complex, called prothrombinase, constituted by activated Factor X (FXa), activated Factor V (FVa), phospholipids and calcium. It is prothrombinase which activates prothrombin to thrombin, enabling the conversion of soluble fibrinogen to insoluble fibrin which forms the clot.

The extrinsic pathway involves the intervention of the FVII present in the plasma. However, the latter must previously be activated to FVIIa in order to initiate the coagulation cascade. FVIIa alone (not complexed with tissue factor) exhibits a low proteolytic activity. This activity is potentialized when the FVIIa is complexed with tissue factor (TF), a protein associated with phospholipids, which is released during formation of the vascular lesion. The FVIIa-TF complex converts the Factor X to Factor Xa in the presence of calcium ions. The FVIIa-TF complex also converts FIX to FIXa.

In return, Factors IXa and Xa activate FVII to FVIIa. Factor Xa complexed with Factor Va and phospholipids (prothrombinase) converts the prothrombin to thrombin. The thrombin acts on the fibrinogen, converting it to fibrin and also carries out other activities, among which is the activation of Factor V to Factor Va and FVIII to FVIIIa. In the presence of calcium, thrombin also activates Factor XIII to Factor XIIIa which allows the consolidation of the fibrin clot. Although in the extrinsic coagulation pathway, FIX is activated to FIXa by the FVIIa/TF complex, in the intrinsic coagulation pathway, FIXa is generated from FIX by FIXa, itself activated by Factor XII activated by the contact of the blood with an electronegative surface such as the sub-endothelium.

FVIIa, a glycoprotein dependent on vitamin K, thus plays a significant role in the coagulation mechanisms, resulting in the formation of a blood clot. FVIIa has the advantage of being able to act locally in the presence of tissue factor released after a lesion of tissues causing hemorrhaging, even in the absence of Factor VIII or IX. This is why FVIIa has for many years been used to remedy certain coagulation disorders that manifest themselves by bleeding.

The first approach was to obtain FVIIa from plasma. However the production of FVIIa from plasma is limited by the availability of the source of supply and this use of plasma presents risks of transmission of pathogenic agents, such as for example the prion and viruses. These problems were overcome by Novo Nordisk Pharmaceuticals with the development of a recombinant FVIIa (rFVIIa) which is a glycoprotein structurally similar to plasma FVIIa.

The principal therapeutic indication for rFVIIa (in the USA, EU and Japan) relates to the treatment of spontaneous or surgical bleeding in type A hemophiliacs having developed anti-Factor VIII antibodies and type B hemophiliacs having developed anti-Factor IX antibodies. In Europe, it is also indicated for its use in patients with a congenital FVII deficiency and in patients suffering from Glanzmann's thrombasthenia. Moreover, many publications report the efficacy of rFVIIa in the control of hemorrhaging during surgical operations, in patients who have neither a congenital coagulation factor deficiency nor thrombasthenia.

This increasingly wide use of FVIIa has led to updating of the methods in order to
(1) measure the FVIIa activity
(2) determine the FVIIa concentration
(3) measure the activated FVII level.
   The best-known methods for detecting FVII activity are measuring the coagulation time, PTT (partial thromboplastin time), aPTT (activated partial thromboplastin time), TEG (thromboelastograph) and TGT (thrombin generation test). These methods make it possible to detect the FVIIa activity but to date do not allow the level of activated FVII in a sample to be directly measured.

A commercial kit for the immunological assay of FVIIa is available (IMUBIND Factor VII ELISA kit) but the experimental conditions for implementing this technique are difficult to master. In fact this kit is complex to use and is characterized by an extremely narrow dynamic range, an extremely limited linear detection range and most important of all, needs to work at a temperature of +4° C.

Other methods for measuring the FVIIa concentration are described in the literature, such as measuring its proteolytic activity with the use of truncated recombinant TF (Staclot VIIa-rTF, Stago) (U.S. Pat. No. 5,472,850, U.S. Pat. No. 5,741,658, WO 1992/018870, U.S. Pat. No. 5,750,358, U.S. Pat. No. 5,741,658, U.S. Pat. No. 5,472,850, EP 0 641 443) or measuring the concentration of an FVIIa-antithrombin complex (WO 03/004694). However, these methods are not very accurate and are difficult to implement. In fact, as these methods allow only a few samples to be processed at the same time, at low FVIIa concentrations, the clot formed is inadequate to implement the method correctly. Moreover, the methods of fluorogenic or chromogenic assay of the FXa generated by the FVIIa have also been found unsuitable for measuring the FVIIa concentration as they do not allow the effect of the FVII to be differentiated from that of the FVIIa.

Among the methods used by some biologists in order to assess the efficacy of a treatment with FVIIa, thrombo-elastography is sometimes used. This method consists of measuring the physical properties of a total blood by mechanically analyzing the formation of the clot as a function of time. According to the parameters extracted from a graph (called a Thromboelastogramme®) generated by the thrombo-elastograph, the clinician can assess the coagulation capability of a patient. Although accurate, this method is tedious, unsuited to routine and repetitive analysis, and difficult to apply to multisampling as it needs to be carried out within an hour after taking the blood. Moreover, this method does not allow the activated-FVII level in a sample to be measured.

The best-known method for measuring the activated FVII level consists of measuring the FVII+FVIIa concentration and the FVIIa concentration independently in order to deduce therefrom the activated FVII level (ratio of FVIIa concentration/FVII+FVIIa concentration). Despite the fact that the measurement of the FVII+FVIIa concentration is accurate, the direct measurements of the FVIIa concentration remain inaccurate and difficult to carry out. Thus there is a genuine need for an available method that is effective and easy to use for measuring the activated FVII level in a sample, in particular when the sample contains a mixture of non-activated Factor VII and activated Factor VII.

### SUMMARY

Surprisingly, the Applicant has found that using a plasma deficient in FVII and in at least one other factor chosen from FVIII, FIX and FXI makes it possible to measure the activated FVII level in a sample in a way that is reliable, reproducible and easy to implement, in particular when the sample contains non-activated Factor VII and activated Factor VII. The experimental conditions implemented in the method of the invention thus make it possible to establish a correlation between the activated FVII level in a sample and certain parameters characteristic of a thrombin generation test (TGT) and of the resulting thrombogram. Such a correlation makes it possible to determine the activated FVII level in a sample to be tested, by comparing the thrombogram parameters of said sample with those of “standard” thrombograms obtained from compositions comprising known activated FVII levels.

The present invention therefore relates to a method for measuring the activated Factor VII level in a test sample, comprising the steps consisting of:
a) mixing said test sample with a plasma deficient in Factor VII (FVII) and deficient in at least one other factor chosen from Factor VIII (FVIII), Factor IX (FIX) and Factor XI (FXI), the mixture of test sample+plasma] having a final FVII+FVIIa concentration ranging from 10 pM to 80 pM,
b) adding components initiating the thrombin generation reaction;
c) obtaining a thrombogram by carrying out a thrombin generation test (TGT) on the mixture of step b);
d) comparing at least one of the thrombogram parameters of step c) to a homologous parameter of standard thrombograms established on the basis of standard samples the activated Factor VII level of which is known and varies between each standard sample;
e) deducing from step d) a measurement of the activated Factor VII level in the test sample.
   The method of the present invention optionally comprises an additional step f) consisting of calculating the concentration of the activated Factor VII in said test sample from the level determined in step e).

Preferably, the standard thrombograms are obtained by carrying out a thrombin generation test on a mixture containing
(i) a standard sample the activated Factor VII level of which is known,
(ii) a plasma deficient in FVII and deficient in at least one other factor chosen from FVIII, FIX and FXI, the final concentration of the mixture of standard sample+plasma deficient in FVII and deficient in at least one other factor chosen from FVIII, FIX and FXI being substantially identical to that of the test sample+plasma deficient in FVII mixture and deficient in at least one other factor chosen from FVIII, FIX and FXI, and
(iii) components initiating the thrombin generation reaction.
   Advantageously, the compared thrombogram parameter is chosen from the lag time, the time to peak and the velocity when said plasma is deficient in FVII and FIX, or deficient in FVII and FXI; and from the lag time and the time to peak when said plasma is deficient in FVII and FVIII.

Preferentially, the test sample+plasma and standard sample+plasma mixtures are carried out using the same plasma deficient in FVII and deficient in at least one other factor chosen from FVIII, FIX and FXI. Advantageously, the components initiating the thrombin generation comprise a tissue factor (TF), phospholipids, and Ca²⁺, the final concentration of said tissue factor in the sample+plasma+initiating components mixture being comprised within the range 1 to 10 pM, the final concentration of said phospholipids in the sample+plasma+initiating components mixture being comprised within the range 0.1 to 5 μM and the final concentration of Ca²⁺ in the sample+plasma+initiating components mixture being comprised within the range 14 to 18 mM. Advantageously, the activated Factor FVII the level of which is measured is of plasma origin (pFVIIa), recombinant origin (rFVIIa) or transgenic origin (TgFVIIa).

In a particular embodiment of the invention, the test sample is a sample of milk from a transgenic mammal or a serum-free cell culture medium. The present invention also relates to the use of a plasma deficient in FVII and in at least one other factor chosen from FVIII, FIX and FXI for measuring the activated FVII level in a test sample.

### DETAILED DESCRIPTION

Within the context of the present invention, by “Factor VII”, or “FVII”, is meant non-activated Factor VII corresponding to the single-strand proenzyme which cannot trigger coagulation. By “activated Factor VII” or “activated FVII”, or FVIIa is meant the single-strand protein (enzyme), comprising a heavy chain and a light chain linked together by a disulphide bridge, resulting from the cleavage of the FVII (proenzyme), and which demonstrates the ability to trigger blood coagulation. By “FVII+FVIIa”, is meant the sum of the concentrations or the sum of the quantities of FVII and FVIIa present in a sample of interest. The sum of the quantities or the concentrations of FVII and FVIIa can be measured, for example, by immunological assay using commercially available kits such as ASSERACHROM® VII: Ag from Diagnostica Stago (Reference 00241).

By “activated Factor VII level” or “FVIIa level”, is meant the ratio between the quantity or the concentration of activated Factor VII (FVIIa) in a sample of interest, and the sum of the quantities or the concentrations, respectively, of Factor VII and activated Factor VII (FVII+FVIIa) in this same sample of interest. The activated FVII level will be equal to 1 (i.e. 100%) for a sample containing only FVIIa but not containing FVII, this level will be equal to 0.5 (i.e. 50%) for a sample containing as much FVIIa as FVII, and it will be equal to 0 (i.e. 0%) for a sample containing FVII only (not containing FVIIa).

By “test sample”, is meant a sample containing FVII, activated FVII or a mixture of these, but the activated FVII level of which is unknown. Advantageously, the test sample is purified from blood or plasma or originates from a bodily fluid, purified or not, such as, for example, mammal's milk, a culture medium or a cell homogenate. In a particular embodiment, the test sample is a sample of mammal's milk, in particular a sample of milk from a transgenic mammal producing FVII and/or FVIIa in its milk. In a further embodiment, the test sample is a serum-free cell culture medium. According to an embodiment of the invention, the sample to be tested is a sample, therapeutic or not, containing FVIIa from plasma (pFVIIa), recombinant (rFVIIa) or transgenic (TgFVIIa), and FVII, or a mixture of these in liquid or freeze-dried form.

By “standard sample”, is meant a sample, the activated FVII level of which is known and/or chosen, incorporating, for example, suitable quantities or concentrations of international standard FVII (Blood Coagulation Factor VII Concentrate Human, NIBSC reference 97/592) or international standard activated FVII (Blood Coagulation Factor VIIa Concentrate Human, NIBSC reference 89/688), or a mixture of these, in a solution of interest, in order to obtain a desired level of activated FVII. The standard sample can also be obtained from blood or plasma or originate from a bodily fluid, purified or not, such as, for example, mammal's milk, a culture medium or a cell homogenate. The plasma, as used within the context of the present invention, is of animal origin, preferably mammal and preferentially human.

Within the meaning of the present invention, the expressions “depleted of” or “deficient in” reflect the same meaning and can be used as alternatives in order to denote a depletion of a solution of interest (for example a plasma) of a compound (for example a blood coagulation factor), until the presence of the latter becomes undetectable. The expression “plasma deficient in FVII and in at least one factor chosen from FVIII, FIX or FXI” therefore means that the respective concentration of each of these Factors FVII, FVIII, FIX or FXI in the plasma considered is below their detection threshold when their concentration is measured by the assay methods known to a person skilled in the art. By way of examples of assay methods, there can be mentioned those which use commercial kits or reagents (for example, ASSERACHROM® VII: Ag from Diagnostica Stago (Reference 00241) or Factor VII ELISA Set from KORDIA (Reference FVII-EIA).

Preferably, the detection threshold of FVII in the plasma is approximately 1 mUl/ml (0.5 ng/ml), a concentration below which the FVII cannot be detected. Preferably, the detection threshold of FVIII in the plasma is approximately 10 mUl/ml (1 ng/ml), a concentration below which the FVIII cannot be detected. Preferably, the detection threshold of FIX in the plasma is approximately 0.2 mUl/ml (1 ng/ml), a concentration below which the FIX cannot be detected. Preferably, the detection threshold of FXI in the plasma is approximately 0.5 mUl/ml (2.5 ng/ml), a concentration below which the FXI cannot be detected.

The techniques for depleting the plasma of a factor of interest include all techniques known to a person skilled in the art. By way of examples of depletion techniques, can be mentioned in particular immunodepletion, chemical depletion, as well as the combination of the latter.

Immunodepletion consists of using antibodies specifically targeted against an antigen contained in a solution with the object of substantially depleting said solution in the antigen considered. The antibodies used for carrying out immunodepletion can be polyclonal and/or monoclonal, originating from a single or different cell clones. The antibodies used can be targeted directly against the antigen that it is desired to eliminate or against a protein which binds to this antigen.

A plasma depleted of FVII, FIX or FXI can thus be obtained using respectively, anti-FVII, anti-FIX or anti-FXI antibodies. A plasma deficient in FVIII can be obtained using anti-FVIII antibodies or antibodies targeted against von Willebrand factor, a plasma protein which transports FVIII in the blood. A plasma depleted of FVIII can also be obtained by chemical depletion, using EDTA (ethylene-diamine-tetraacetic acid) insofar as FVIII is a Ca²⁺-dependent factor. The EDTA is then eliminated by methods well known to a person skilled in the art, for example by dialysis.

Advantageously, the plasma used to implement the present invention is a plasma deficient in Factor VII and in at least one other factor chosen from FVIII, FIX and FXI. Advantageously, the plasma used to implement the present invention is prepared from a plasma from a type-A hemophiliac, naturally deficient in FVIII, from a plasma from a type-B hemophiliac, naturally deficient in FIX, or from a plasma originating from patients exhibiting a total Factor XI deficiency, said plasmas, being naturally deficient in FVIII, FIX or FXI, are then FVII-depleted, by an immunological or chemical method such as those described above. In a further embodiment of the invention, the plasma used is prepared from a normal plasma which is initially FVII-depleted then, subsequently, depleted of FVIII and/or FIX and/or FXI.

Within the meaning of the present invention, by “components initiating the thrombin generation reaction” or “initiating components”, is meant the indispensable components allowing the generation of thrombin from prothrombin to be started. The components initiating the thrombin generation reaction essentially comprise a calcium ion source (Ca²⁺), a phospholipidic agent and tissue factor (TF), in adequate concentrations to trigger the thrombin generation reaction.

A suitable source of calcium ions within the context of the present invention corresponds to any biologically compatible source of calcium ions, such as CaCl₂. The source of Ca²⁺ can be added to the sample/plasma mixture extemporaneously with the other components initiating the thrombin generation reaction or in a deferred manner, i.e. after the addition of the other components initiating the thrombin generation reaction. Within the meaning of the present invention, by adequate concentration of calcium ions is meant a final concentration of calcium ions in the sample+plasma+initiating components mixture comprised within the range from 14 to 18 mM, and in particular equal to 16.7 mM.

Phospholipidic agents suitable for use in the present invention can be in the form of concentrate or freeze-dried product and consist, preferably, of a mixture comprising a majority quantity of phosphatidylcholine and phosphatidylserine or containing exclusively phosphatidylcholine and phosphatidyl-serine. Within the meaning of the present invention, by adequate concentration of phospholipidic agents is meant a final concentration of phospholipidic agents in the sample+plasma+initiating components mixture comprised within the range from 0.1 to 5 μM, in particular 0.5 to 2 μM, and more particularly equal to 1 μM.

A tissue factor (TF) appropriate for use in the present invention can be chosen from the group constituted by any native, plasma, recombinant or transgenic tissue factor, any modified tissue factor, including any truncated tissue factor having lost its function of activating Factor VII to Factor VIIa, providing that said modified tissue factor has preserved, even partially, its capacity to act as cofactor of the enzymatic activity of Factor VIIa. A suitable modified tissue factor can for example be transmembrane domain-deleted such as the tissue factor of the STACLOT kit commercially available from Diagnostica Stago (Reference 00281). Within the meaning of the present invention, by adequate concentration of tissue factor is meant a final concentration of tissue factor in the sample+plasma+initiating components mixture comprised within the range from 1 to 10 pM, in particular 4 to 6 pM, and more particularly equal to 5 pM.

Within the context of the invention, the test sample, the standard sample, the plasma deficient in FVII and at least one other factor chosen from FVIII, FIX or FXI, and/or the components initiating the thrombin generation reaction can be in liquid or freeze-dried form. When they are in the freeze-dried form, these compounds can advantageously be placed in suspension, prior to implementation of the method according to the invention, in a suitable aqueous solvent, such as purified water for injection (WFI).

The Applicant has therefore developed a method for measuring the activated Factor VII level in a test sample based on specific experimental conditions which make it possible to overcome the disadvantages resulting from the presence of non-activated FVII in the test sample. The first step of this method consists of mixing a test sample containing an unknown activated FVII level with a plasma deficient in FVII and deficient in FVIII, and/or FIX and/or FXI, such that the final FVII+FVIIa concentration in the resulting mixture is comprised within the range from 10 pM to 80 pM. This specific combination of the properties linked to the nature of the plasma and the concentration range used makes it possible to implement the method for measuring the activated FVII level according to the invention.

Components initiating the thrombin generation reaction are then added to the test sample+plasma mixture with the aim of triggering the cleavage reaction which leads to the thrombin generation from the prothrombin contained in the plasma. A thrombin generation test is then carried out. The thrombin generation test (TGT) is a test known to a person skilled in the art (Thrombin generation assays: accruing clinical relevance, H. C Hemker, R. Al Dieri & S. Béguin, Curr. Opin. Hematol., 2004, 11, 170-5) which makes it possible to measure, in a continuous manner, the quantity of thrombin generated and the time necessary for generating this thrombin when a sample of interest is placed in contact with components initiating the thrombin generation reaction.

The thrombin generation test starts when the sample of interest (or a solution comprising the latter) is placed in contact with components initiating the reaction. This initial time corresponding to the start of the thrombin generation test is called t₀. The thrombin generated is then revealed by the use of a revealing agent, preferably by using a fluorogenic agent, the degradation of which by thrombin causes the appearance of a fluorescent compound, or by using a chromogenic agent. Advantageously, the fluorogenic agent or the chromogenic agent is added to the sample+plasma mixture at same time as the components initiating the thrombin generation reaction.

The fluorescence resulting from the degradation of the fluorogenic agent by the newly generated thrombin is detected by a measuring device such as a fluorimeter. Preferably, the fluorimeter used is provided with means of recording or means of plotting the variation in fluorescence over time. The data collected by the fluorimeter make it possible to establish the variation curve of the fluorescence over time, called a thrombogram.

Four derived parameters can be measured from a thrombogram:
the peak height, expressed in nM of thrombin, corresponds to the maximum concentration of thrombin generated at a time tₘₐₓ during the reaction;
the lag time, expressed in minutes, corresponds to the time elapsed between the start of the thrombin generation test (t₀) and the appearance of the thrombin;
the time to peak, expressed in minutes, corresponds to the time elapsed between the start of the TGT (t₀) and the time tₘₐₓ corresponding to the maximum thrombin generated;
the velocity, expressed in nM of thrombin formed/min, corresponds to the height of the peak divided by the difference between the time to peak tₘₐₓ and the lag time.
   Advantageously, these parameters are provided directly by the device used for measuring the thrombin formation. The higher the FVIIa level in the sample of interest, the more rapidly the thrombin is generated, the shorter the time to peak, and the higher the velocity.

In order to determine the activated FVII level of the test sample, standard thrombograms are obtained from standard samples containing a known activated FVII level. As described above for the test sample, at least two standard samples containing different levels of activated FVII are mixed with a plasma deficient in FVII and deficient in at least one factor chosen from FVIII, FIX and FXI. Components initiating the thrombin generation reaction are then added to the standard sample+plasma mixture in order to start the thrombin generation test and in order to obtain the standard thrombograms corresponding to each standard sample.

The FVII+FVIIa concentration of the standard sample+plasma mixture is comprised between 10 pM and 80 pM. Preferably, the FVII+FVIIa concentration of the standard sample+plasma mixture is substantially identical to the FVII+FVIIa concentration of the test sample+plasma mixture. Advantageously, the plasma mixed with the standard samples is identical to that which was mixed with the test sample.

From the standard thrombograms obtained, an interpolation is carried out between the level of activated FVII contained in each standard sample and one of the parameters derived from a thrombogram. The interpolation carried out can be of linear, geometric, cubic, polynomial, lagrangian or newtonian type. Advantageously, the interpolation carried out corresponds to the variation of the lag time as a function of the activated FVII level in the standard samples, the variation of the time to peak as a function of the activated FVII level in the standard samples or the variation of the velocity as a function of the activated FVII level in the standard samples.

The level of activated FVII contained in the test sample is determined by transferring at least one of the parameters derived from the thrombogram of this test sample to a corresponding interpolation carried out from the standard thrombograms. If the parameter transferred is the lag time, the interpolation used will correspond to the variation of the lag time as a function of the activated Factor VII level in the standard samples. If the transferred parameter is the time to peak, the interpolation used will correspond to the variation of the lag time as a function of the activated Factor VII level in the standard samples. Finally, if the transferred parameter is the velocity, the interpolation used will correspond to the variation of the velocity as a function of the activated Factor VII level in the standard samples. The Factor VII level determined from the interpolation therefore corresponds to the activated Factor VII level of the test sample.

The method of the invention can comprise an additional step consisting of calculating the concentration of the activated Factor VII in the test sample from the activated Factor VII level determined from the interpolation. In this particular case, the concentration of activated Factor VII is given by the following formula:

FVIIa concentration=FVIIa level×FVII+FVIIa concentration

The examples below illustrate the invention without limiting its scope.

### BRIEF DESCRIPTION OF THE DRAWINGS

FIG. 1: Standard thrombograms obtained in the presence of a final FVII+FVIIa concentration of 50 pM in the standard sample/plasma mixture, with a plasma deficient in FVII and FVIII, and for levels of activated FVII ranging from 0 to 100% (with 5 pM tissue factor and 1 μM phospholipids).

FIGS. 2 and 2a: Variations in the lag time and the time to peak, respectively, as a function of the activated FVII level in the standard sample, with a plasma deficient in FVII and FVIII, for a final FVII+FVIIa concentration of 50 pM (with 5 pM tissue factor and 1 μM phospholipids).

FIG. 3: Standard thrombograms obtained in the presence of a final FVII+FVIIa concentration of 50 pM in the standard-sample/plasma mixture, with a plasma deficient in FVII and FIX (with 5 pM tissue factor and 1 μM phospholipids).

FIGS. 4, 5 and 6: Variations in the lag time, the time to peak and the velocity, respectively, as a function of the activated FVII level in the standard sample, with a plasma deficient in FVII and FIX, for a final FVII+FVIIa concentration of 50 pM (with 5 pM tissue factor and 1 μM phospholipids).

FIG. 7: Standard thrombograms obtained in the presence of a final FVII+FVIIa concentration of 50 pM in the standard sample/plasma mixture, with a plasma deficient in FVII and FXI (with 5 pM tissue factor and 1 μM phospholipids).

FIGS. 8, 9, 10 and 11: Variations in the lag time, the time to peak, the peak height, and the velocity, respectively, as a function of the activated FVII level in the standard sample, with a plasma deficient in FVIII and FXI, for a final FVII+FVIIa concentration of 50 pM (with 5 pM tissue factor and 1 μM phospholipids).

FIG. 12: Standard thrombograms obtained in the presence of a final FVII+FVIIa concentration of 10 pM in the standard sample/plasma mixture, with a plasma deficient in FVII and FVIII (with 5 pM tissue factor and 1 μM phospholipids).

FIGS. 13, 14, 15 and 16: Variations in the lag time, the time to peak, the peak height, and the velocity, respectively, as a function of the activated FVII level in the standard sample, with a plasma deficient in FVII and FVIII, for a final FVII+FVIIa concentration of 10 pM (with 5 pM tissue factor and 1 μM phospholipids).

FIG. 17: Standard thrombograms obtained in the presence of a final FVII+FVIIa concentration of 80 pM in the standard sample/plasma mixture, with a plasma deficient in FVII and FVIII (with 5 pM tissue factor and 1 μM phospholipids).

FIGS. 18 and 19: Variations in the lag time and the time to peak, respectively, as a function of the activated FVII level in the standard sample, with a plasma deficient in FVII and FVIII, for a final FVII+FVIIa concentration of 80 pM (with 5 pM tissue factor and 1 μM phospholipids).

### EXAMPLES

### Example 1

### Preparation of a Plasma Deficient in FVII

Polyclonal antibodies produced in the rabbit, targeted against purified human plasma FVII were coupled to CNBr-activated sepharose (Pharmacia), then 2 mL of the gel obtained were placed in a column. The column was equilibrated with 25 mL of equilibration buffer (0.15 M NaCl, 10 mM citrate, pH 7.4). Then, 6 mL of human plasma was passed several times over the column. Under these conditions, the FVII remained fixed on the column and the eluate was recovered (plasma deficient in FVII). The column was regenerated by eluting the fixed FVII with 20 mL of regeneration buffer (50 mM NaCl; 0.1 M glycine, pH 2.4) then the column was re-equilibrated with 20 mL of equilibration buffer (10 mM citrate; 0.15 M NaCl, pH 7.4).

### Example 2

### Preparation of a Plasma Deficient in FVII and FVIII, FIX or FXI

Polyclonal antibodies produced in the rabbit, targeted against purified human plasma FVII were coupled to CNBr-activated sepharose (Pharmacia), then 2 mL of the gel obtained was placed in a column. The column was equilibrated with 25 mL of equilibration buffer (0.15 M NaCl, 10 mM citrate, pH 7.4). Then, 6 mL of commercial plasma already depleted of FVIII, FIX or FXI, each obtained from Diagnostica Stago, were passed several times over the column. Under these conditions, the FVII remained fixed on the column and the eluate was recovered (plasma doubly deficient in FVII and FVIII, FIX or FXI). The column was regenerated by eluting the fixed FVII with 20 mL of regeneration buffer (50 mM NaCl; 0.1 M glycine, pH 2.4) then the column was re-equilibrated with 20 mL of equilibration buffer (10 mM citrate; 0.15 M NaCl, pH 7.4).

### Example 3

### Preparation of a Standard Thrombogram from a Plasma Deficient in FVII and FVIII, for an FVII+FVIIa Concentration of 50 pM

An adequate volume of a sample of international standard FVII (SI-FVII) supplied by NIBSC and/or international standard FVIIa (SI-FVIIa) also supplied by NIBSC is taken in order to obtain a standard sample containing a known activated FVII level, which is added to 80 μL of plasma deficient in FVII and FVIII, (plasma deficient in FVIII obtained from Diagnostica Stago or being a type A hemophiliac plasma, which was then FVII-depleted as in Example 2), in order to obtain a mixture which contains a fixed activated FVII level comprised between 0% and 100% for an FVII+FVIIa concentration comprised between 10 pM and 80 pM. 20 μL of factors initiating the thrombin generation reaction (Ca²⁺, phospholipids and TF) are added to the mixture comprising the plasma and the sample, at final concentrations of 5 pM TF, 1 μM phospholipids, (Diagnostica Stago 86195 reagent diluted 1:4) and 16.7 mM Ca², and 20 μL of thrombin-specific fluorogenic agent (Diagnostica Stago 86197 Fluca kit reagent).

The TGT standard curves (standard thrombograms) were established for fixed known activated FVII levels comprised between 0% and 100%, so as to obtain standard thrombograms providing the various parameters (lag time, peak height, time to peak and velocity). The thrombograms are established using a fluorimetric device for measuring the thrombin formation time (Fluoroskan−Thermo Electron) equipped with software for producing the thrombograms (Thrombinoscope BV), for an excitation wavelength of 390 nm and a transmission wavelength of 460 nm.

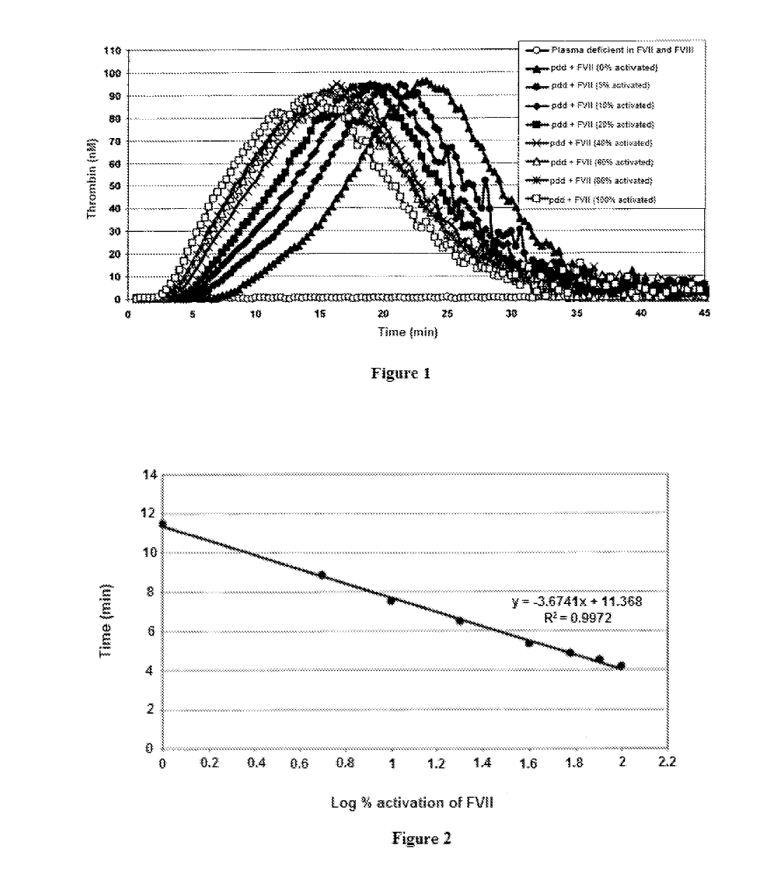

FIG. 1 shows the standard thrombograms obtained in the presence of 50 pM of final FVII+FVIIa in the plasma/sample mixture and for variable activated FVII levels ranging from 0 to 100%. A reduction in the lag time and thrombin formation time at the peak as a function of the increase in the activated FVII level in the sample is observed. The time to peak reaches a limit that can be estimated at 14 minutes for an activated factor level of 100%. It is noted that a plasma deficient in FVII and FVIII without added FVII or FVIIa does not cause thrombin formation.

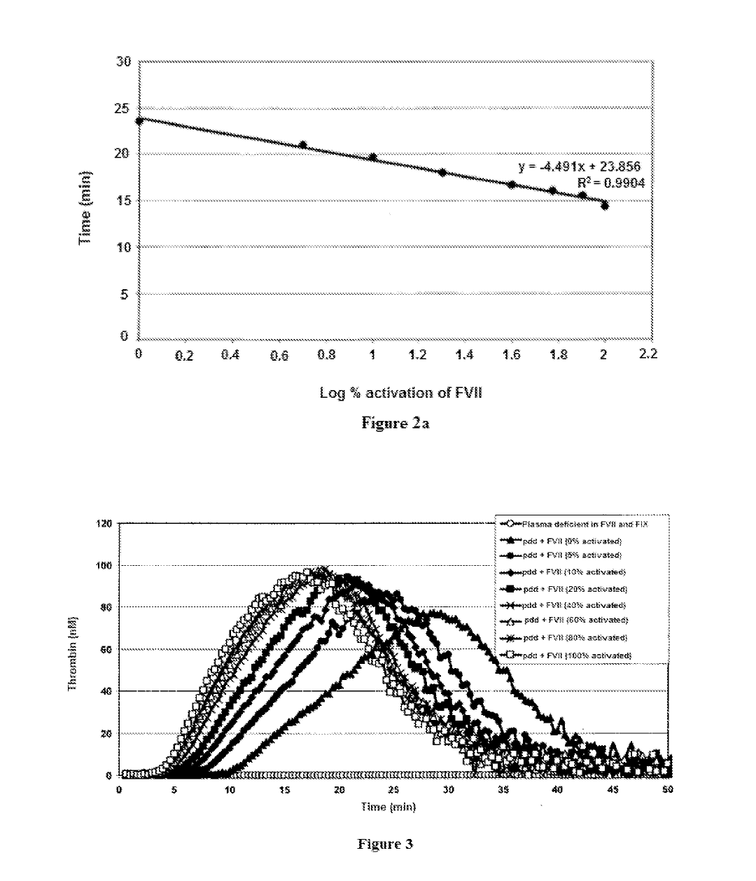

FIGS. 2 and 2a respectively show the variations in the lag time and the time to peak, as a function of the activated FVII level in the sample. The results obtained demonstrate that it is possible to establish a correlation between the activated FVII level in the sample and the different parameters deduced from the thrombograms obtained for an FVII+FVIIa concentration of 50 pM in the plasma/sample mixture.

### Example 4

### Preparation of a Standard Thrombogram from a Plasma Deficient in FVII and FVIX, for an FVII+FVIIa Concentration of 50 pM

The experiment in Example 3 is repeated, but using a reactive plasma deficient in FIX, from Diagnostica Stago, which was then FVII-depleted according to the procedure in Examples 1 and 2. FIG. 3 shows the thrombograms obtained in the presence of 50 pM of FVII+FVIIa in the plasma/sample mixture, said plasma being deficient in FVII and FIX. A reduction in the lag time and thrombin formation time at the peak as a function of the increase in the activated FVII level in the sample is observed. The time to peak reaches a limit that can be estimated at 16 minutes for an activated FVII level of 100%. It is noted that a plasma deficient in FVII and FVIX without added FVII or FVIIa does not cause thrombin formation.

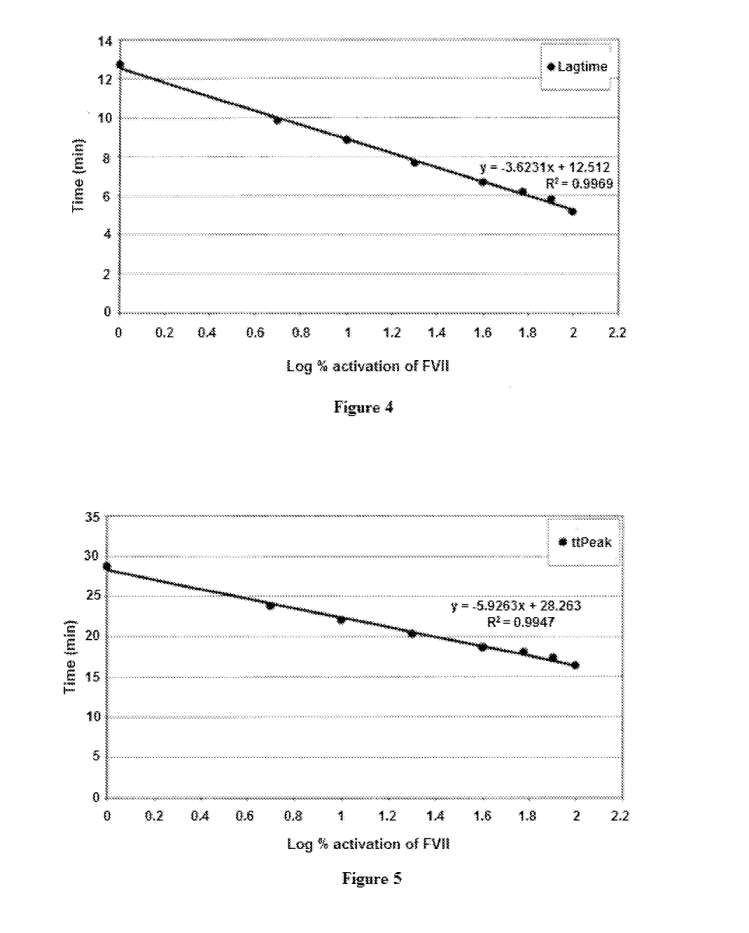

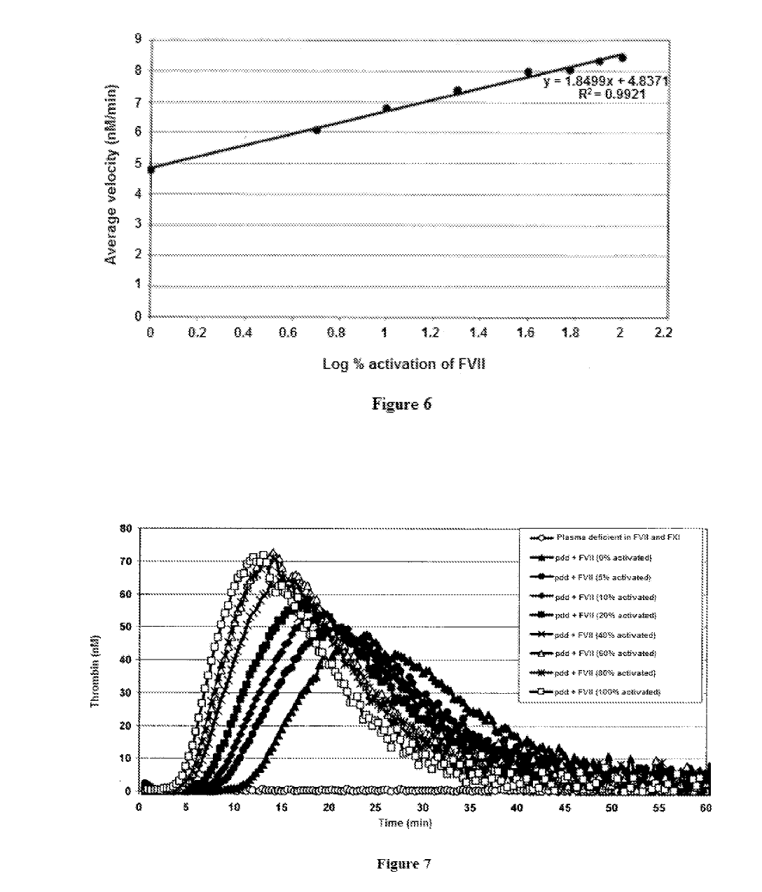

FIGS. 4, 5, and 6 respectively show the variations in the lag time, the time to peak and the velocity, as a function of the activated FVII level in the sample. The results obtained demonstrate that it is possible to establish a correlation between the activated FVII level in the sample and the different parameters deduced from the thrombograms obtained in the presence of 50 pM of FVII+FVIIa in the plasma/sample mixture, said plasma being deficient in FVII and FIX.

### Example 5

### Preparation of a Standard Thrombogram from a Plasma Deficient in FVII and FXI, for an FVII+FVIIa Concentration of 50 pM

The experiment in Example 3 is repeated, but using a reactive plasma deficient in FXI, obtained from Diagnostica Stago, which was then FVII-depleted according to the procedure in Examples 1 and 2. FIG. 7 shows the thrombograms obtained in the presence of 50 pM of FVII+FVIIa in the plasma/sample mixture, said plasma being deficient in FVII and FXI. A reduction in the lag time and thrombin formation time at the peak as a function of the increase in the activated FVII level in the sample is observed. The time to peak reaches a limit that can be estimated at 12 minutes for an activated FVII level of 100%. It is noted that a plasma deficient in FVII and FXI without added FVII or FVIIa does not cause thrombin formation.

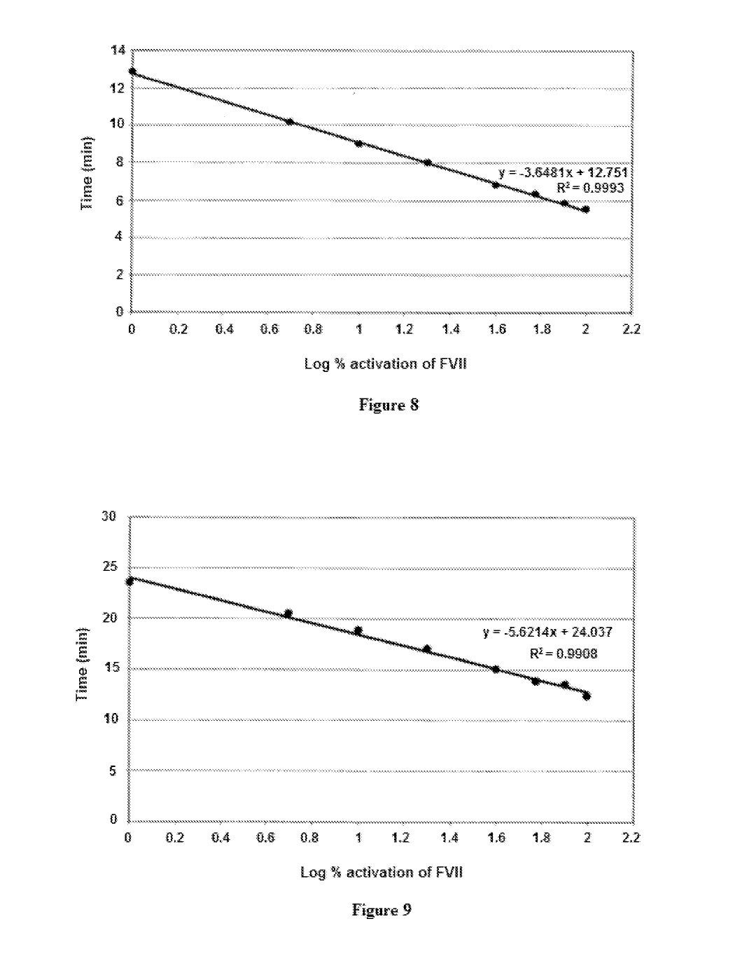

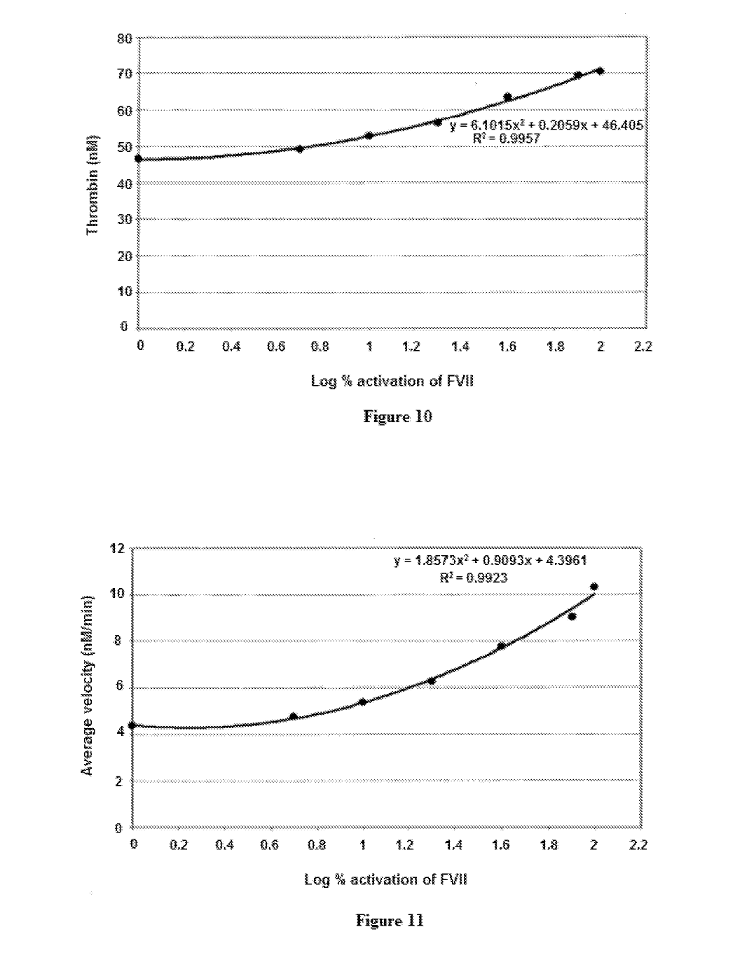

FIGS. 8, 9, 10 and 11 respectively show the variations in the lag time, the time to peak, the peak height and the velocity, as a function of the activated FVII level in the sample. The results obtained demonstrate that it is possible to establish a correlation between the activated FVII level in the sample and the different parameters deduced from the thrombograms obtained in the presence of 50 pM of FVII+FVIIa in the plasma/sample mixture, said plasma being deficient in FVII and FXI.

### Example 6

### Preparation of a Standard Thrombogram from a Plasma Deficient in FVII and FVIII, for an FVII+FVIIa Concentration of 10 pM

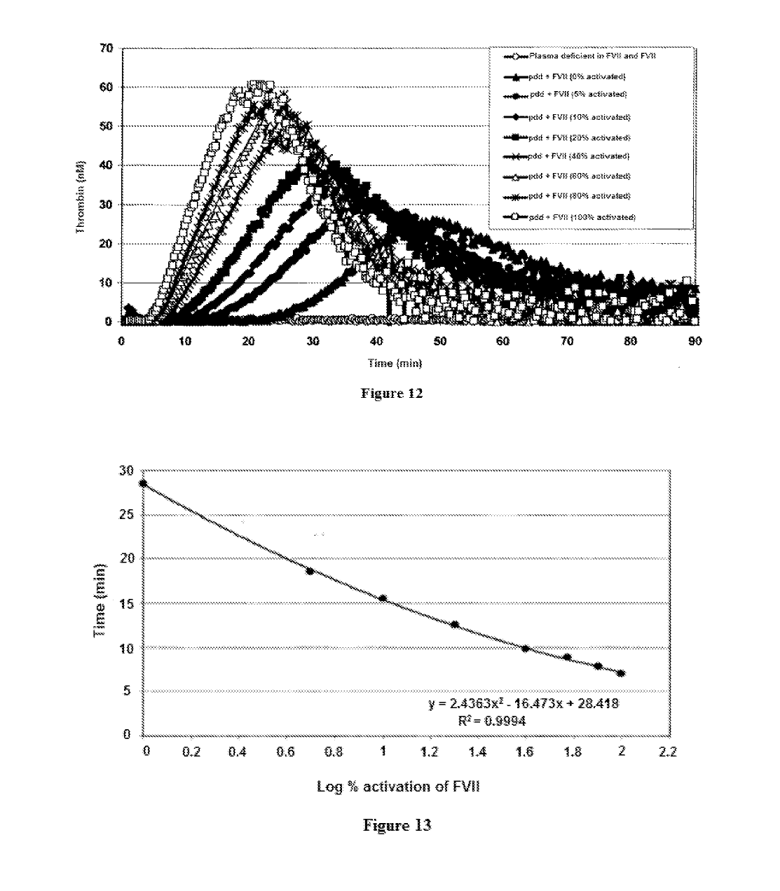

The experiment of Example 3 is repeated, but using a final FVII+FVIIa concentration of 10 pM. FIG. 12 shows the thrombograms obtained in the presence of 10 pM of FVII+FVIIa in the plasma/sample mixture, said plasma being deficient in FVII and FVIII. A reduction in the lag time and thrombin formation time at the peak as a function of the increase in the activated FVII level in the sample is observed. The time to peak reaches a limit that can be estimated at 21 minutes for an activated FVII level of 100%. It is noted that a plasma deficient in FVII and FVIII without added FVII or FVIIa does not cause thrombin formation.

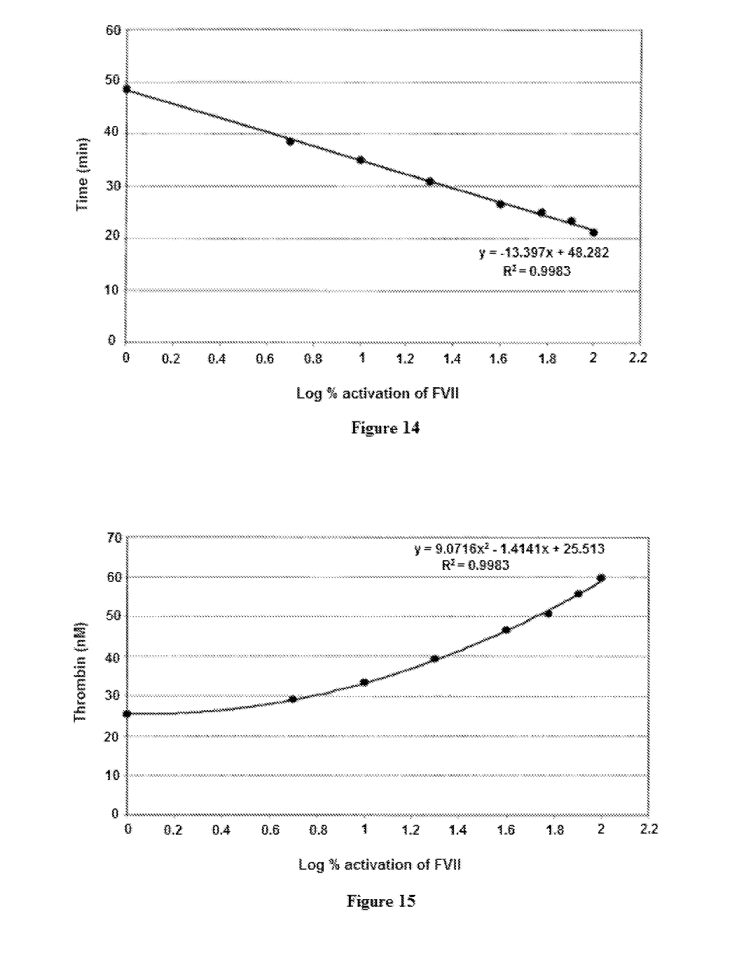

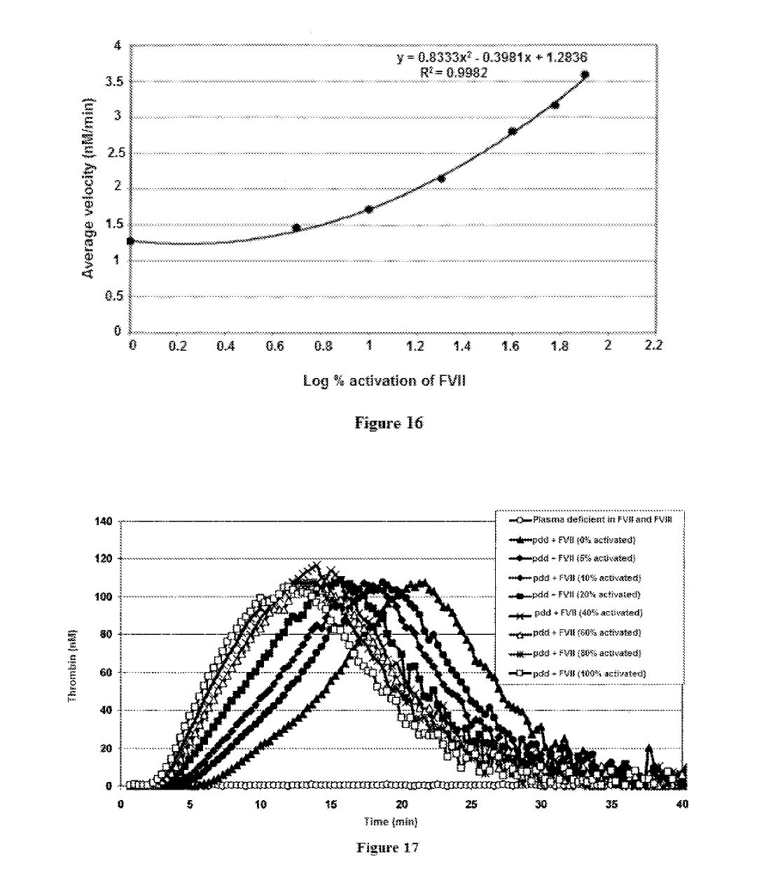

FIGS. 13, 14, 15 and 16 respectively show the variations in the lag time, the time to peak, the peak height and the velocity, as a function of the activated FVII level in the sample. The results obtained demonstrate that it is possible to establish a correlation between the activated FVII level and the different parameters deduced from the thrombograms obtained in the presence of 10 pM of FVII+FVIIa in the plasma/sample mixture, said plasma being deficient in FVII and FVIII.

### Example 7

### Preparation of a Standard Thrombogram from a Plasma Deficient in FVII and FVIII, for an FVII+FVIIa Concentration of 80 pM

The experiment of Example 3 is repeated, but using a final FVII+FVIIa concentration of 80 pM. FIG. 17 shows the thrombograms obtained in the presence of 80 pM of FVII+FVIIa in the plasma/sample mixture, said plasma being deficient in FVII and FVIII. A reduction in the thrombin formation time at the peak as a function of the increase in the activated FVII level in the sample is observed. The time to peak reaches a limit that can be estimated at 12 minutes for an activated FVII level of 100%. It is noted that a plasma deficient in FVII and FVIII without added FVII or FVIIa does not cause thrombin formation.

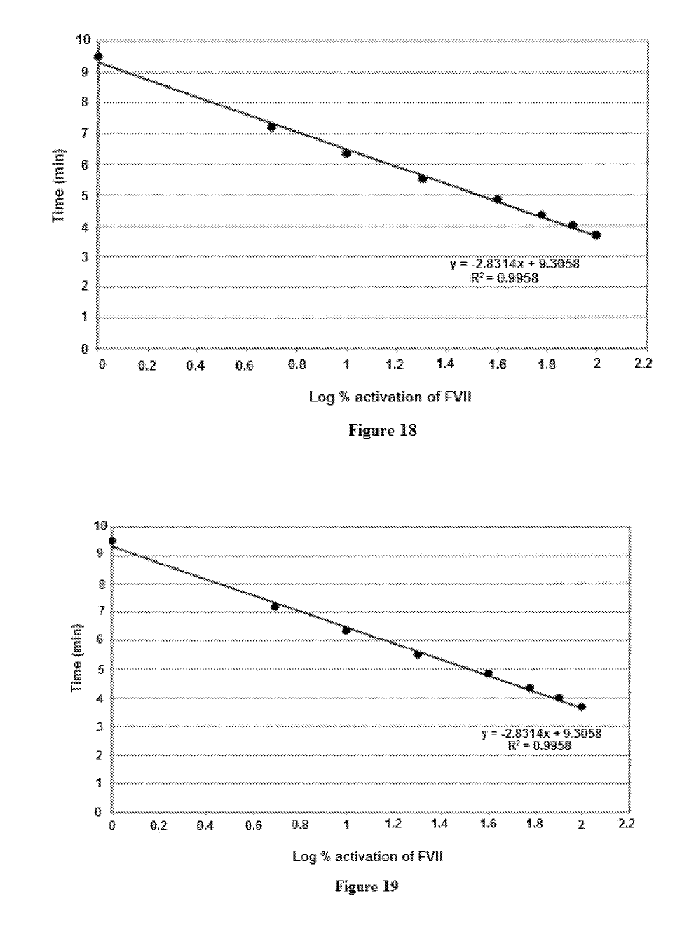

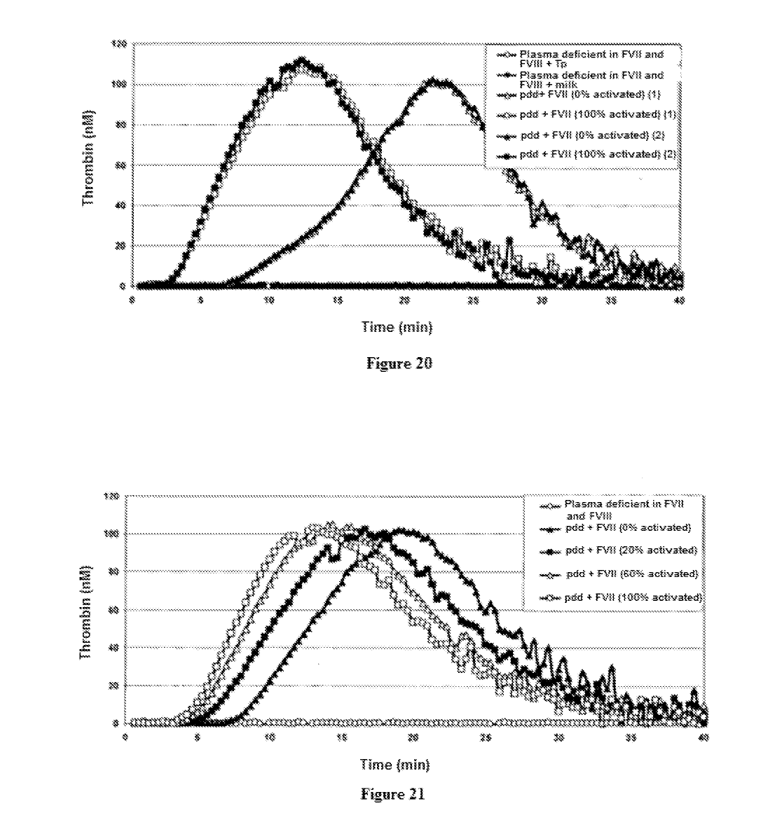

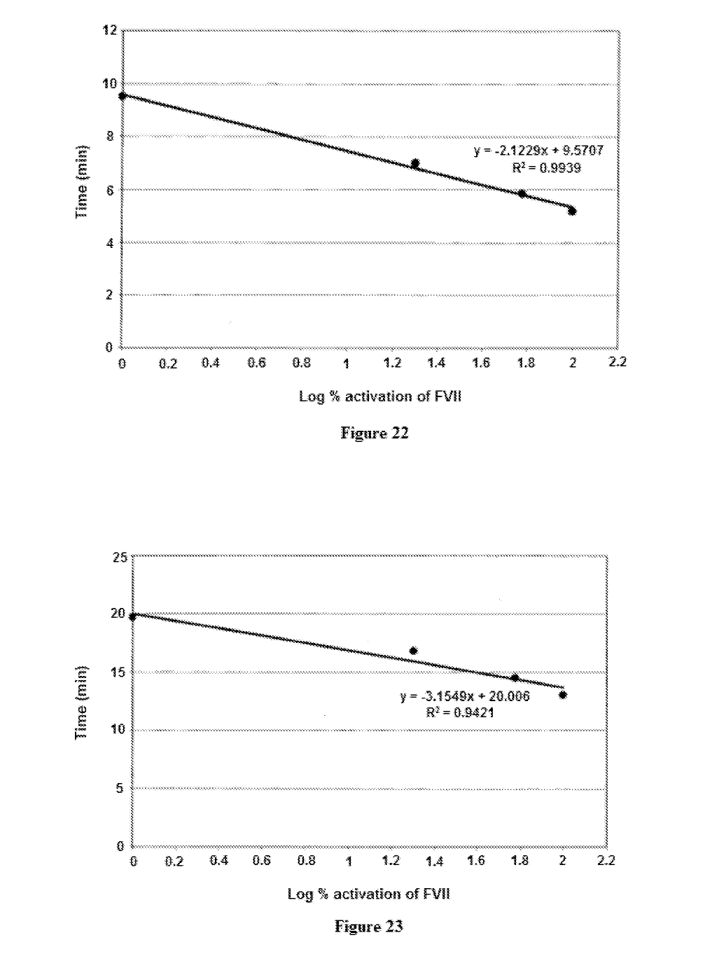

FIGS. 18 and 19 respectively show the variations in the lag time and the time to peak, as a function of the activated FVII level in the sample. The results obtained demonstrate that it is possible to establish a correlation between the activated FVII level in the sample and the different parameters deduced from the thrombograms obtained in the presence of 80 pM of FVII+FVIIa in the plasma/sample mixture, said plasma being deficient in FVII and FVIII.

### Example 8

### Measurement of the Activated FVII Level of a Sample of Rabbit's Milk Containing FVII

The sample to be tested has an unknown activated FVII level and its FVII+FVIIA concentration is 500 pM. A volume of 8 μL of this sample to be tested was mixed with 72 μL of plasma deficient in FVII and FVIII as described previously. A reaction mixture for testing is thus obtained, the volume of which is 80 μL and the FVII+FVIIA concentration, 50 pM. Then, 20 μl of the components initiating the thrombin generation reaction (phospholipids and TF) was added thereto at final concentrations of 5 pM TF, 1 μM phospholipids, (Diagnostica Stago 86195 reagent diluted 1:4), and 20 μl of a thrombin-specific calcareous fluorogenic agent (final concentration of 16.7 mM Ca²⁺) (Diagnostica Stago 86197 Fluca kit reagent).

A TGT was carried out in order to obtain a thrombogram and the corresponding different parameters: lag time and time to peak. At the same time, samples of FVII and FVIIa (international-standard FVII and FVIIa supplied by NIBSC were mixed in order to obtain standard samples, the activated FVII level of which is known and comprised between 0% and 100%. These standard samples were mixed with a plasma deficient in FVII and FVIII as described previously in order to obtain an FVII+FVIIa concentration of 50 pM. Then, components initiating the thrombin generation reaction (phospholipids and TF) were added to the sample/plasma mixture in order to reach final concentrations of 5 pM TF and 1 μM phospholipids (Diagnostica Stago 86195 reagent diluted 1:4). Finally, 20 μl of a thrombin-specific calcareous fluorogenic agent (final concentration 16.7 mM Ca²⁺) (Diagnostica Stago 86197 Fluka kit reagent) were added to the previous mixture. The parameters of standard thrombograms were measured in order to plot standard curves for each of the parameters as a function of the −log of the activated FVII level (see FIGS. 2 and 2a and Table 1). The parameter values obtained from the thrombogram of the mixture containing the sample to be tested were transferred to the different standard curves, making it possible to deduce a measurement of the activated FVII level in the sample to be tested. A lag time of 6 minutes and 30 seconds and a peak time of 18 minutes are obtained, and by transferring these values to the different standard curves, a measurement of the activated FVII level in the sample to be tested, equal to 20%, is deduced.

### Example 9

### Influence of Rabbit's Milk on the Thrombin Generation Test

The experiment in Example 3 is repeated, but pre-diluting the standard sample containing a known activated FVII level in Owren-Koller buffer containing 1% of human serum albumin (OK buffer—1% HSA) or in OK buffer—1% HSA containing rabbit's milk. FIG. 20 shows the standard thrombograms obtained in the presence of 50 pM final FVII+FVIIa in the plasma/sample mixture and for activated FVII levels of 0 and 100%. It is observed that the thrombograms of the sample pre-diluted in OK buffer—1% HSA and the sample pre-diluted in OK buffer—1% HSA containing rabbit's milk are perfectly superimposed, making it possible to deduce that the rabbit's milk has no influence on the TGT. It is noted that a plasma deficient in FVII and FVIII without added FVII or FVIIa does not generate thrombin formation.

FIGS. 21, 22 and 23 respectively show the thrombograms, the variations in the lag time and the time to peak, as a function of the activated FVII level in the Tp OK—1% HSA pre-diluted sample containing rabbit's milk for known fixed activated FVII levels comprised between 0% and 100%. The results obtained demonstrate, as in Example 3, that it is possible to establish a correlation between the activated FVII level in the sample containing rabbit's milk and the different parameters deduced from the thrombograms for an FVII+FVIIa concentration of 50 pM in the plasma/sample mixture.

TABLE 1
Final 50 pM FVII + FVIIa in a plasma deficient in FVII and FVIII

| | FVII activation level | | | | | | | |
|---|---|---|---|---|---|---|---|---|
| | 0% | 5% | 10% | 20% | 40% | 60% | 80% | 100 |
| Lag time (min) | 11.5 | 8.83 | 7.50 | 6.50 | 5.33 | 4.83 | 4.50 | 4.17 |
| Time to | 23.5 | 21.0 | 19.6 | 18.0 | 16.6 | 16.0 | 15.5 | 14.3 |
| peak (min) | | | | | | | | |

(5 pM TF − 1 μM PL)

1. A method for measuring the level of activated Factor VII in a test sample, the method comprising:
a) mixing the test sample with a plasma deficient in Factor VII (FVII) and deficient in at least one other factor chosen from Factor VIII (FVIII), Factor IX (FIX) and Factor XI (FXI), the test sample+plasma mixture having a final FVII+FVIIa concentration ranging from 10 pM to 80 pM;
b) adding components initiating the thrombin generation reaction;
c) obtaining a thrombogram by carrying out a thrombin generation test (TGT) on the mixture of step b);
d) comparing at least one of the thrombogram parameters of step c) to a homologous parameter obtained from standard thrombograms established on the basis of standard samples the activated Factor VII level of which is known and varies between each standard sample; and
e) deducing from step d) a measurement of the activated Factor VII level in the test sample.

2. The method according to claim 1, in which each of the standard thrombograms is obtained by carrying out a thrombin generation test on a mixture comprising:
(i) a standard sample the activated Factor VII level of which is known;
(ii) a plasma deficient in FVII and deficient in at least one other factor chosen from FVIII, FIX and FXI, the final FVII+FVIIa concentration of the standard sample+plasma mixture being substantially identical to that of the test sample+plasma mixture; and
(iii) components initiating the thrombin generation reaction.

3. The method according to claim 1, wherein, in step d), the compared thrombogram parameter is chosen from the lag time, the time to peak and the velocity.

4. The method according to claim 3, wherein the plasma is deficient in FVII and FIX, or deficient in FVII and FXI.

5. The method according to claim 1, wherein, in which, in step d), the compared thrombogram parameter is chosen from the lag time and the time to peak.

6. The method according to claim 5, in which the plasma is deficient in FVII and FVIII.

7. The method according to claim 1, wherein the test sample+plasma and standard sample+plasma mixtures are produced using the same plasma deficient in FVII and deficient in at least one other factor chosen from FVIII, FIX and FXI.

8. The method according to claim 1, wherein the components initiating the thrombin generation comprise a tissue factor (TF), phospholipids, and Ca²⁺, the final concentration of the tissue factor in the sample+plasma+initiating components mixture being comprised within the range 1 to 10 pM, the final concentration of the phospholipids in the sample+plasma+initiating components mixture being comprised within the range from 0.1 to 5 μM and the final concentration of Ca²⁺ in the sample+plasma+initiating components mixture being comprised within the range from 14 to 18 mM.

9. The method according to claim 1, wherein the test sample is a sample of milk from a transgenic mammal or a serum-free cell culture medium.

10. The method according to claim 1, wherein the activated factor FVII the level of which is measured is of plasma origin (pFVIIa), recombinant origin (rFVIIa) or transgenic origin (TgFVIIa).

11. The method according to claim 1, further comprising step f) of calculating the concentration of the activated Factor VII in the test sample from the level determined in step e).

12. A use of a plasma deficient in FVII and in at least one other factor chosen from FVIII, FIX and FXI for measuring the activated FVII level in a test sample.
